(19) United States
(12) Patent Application Publication
(10) Pub. No.: US 2026/0205031 A1
(43) Pub. Date: Jul. 16, 2026

(54) MAGNETICALLY-ACTUATED TRIBOELECTRIC NANOGENERATOR AND WIRELESS POWER TRANSFER APPARATUS USING THE SAME

(71) Applicant: University-Industry Cooperation Group of Kyung Hee University, Yongin-si (KR)

(72) Inventors: Yoonseok Park, Suwon-si (KR); Junyeop Kim, Seoul (KR)

(21) Appl. No.: 19/324,314

(22) Filed: Sep. 10, 2025

(30) Foreign Application Priority Data
Jan. 16, 2025 (KR) 10-2025-0006504

(51) Int. Cl. H02N 1/04; H02J 50/10; A61N 1/378

(52) U.S. Cl. CPC H02N 1/04; H02J 50/10; A61N 1/3787

(57) ABSTRACT

A triboelectric nanogenerator according to an embodiment of the present disclosure includes an electrode, a first polymer material layer with a three-dimensional structure, a second polymer material layer attached to at least a part of the first polymer material layer and in contact or not in contact with the electrode depending on an external magnetic field by including magnetic particles, and a spacer provided between the electrode and the first polymer material layer to form a predetermined separation distance, wherein a voltage peak is generated when the second polymer material layer is in contact with the electrode.

### CROSS-REFERENCE TO RELATED PATENT APPLICATION

This application claims the benefit of Korean Patent Application No. 10-2025-0006504, filed on Jan. 16, 2025, in the Korean Intellectual Property Office, the disclosure of which is incorporated herein in its entirety by reference.

### TECHNICAL FIELD

The present disclosure relates to a triboelectric nanogenerator, and more specifically, to a magnetically-actuated triboelectric nanogenerator and a wireless power transfer apparatus using the same.

The present disclosure was derived from research conducted as part of Research Operation Expense Support of the Ministry of Science and ICT (Project Identification No.: 2710001306, Sub-Project No.: 00211261, Project Management Institution: National Research Foundation of Korea, Research Project Title: 3D Time-Limited Micro Robot Controlled by External Magnetic Field, Project Execution Institution: Kyung Hee University University-Industry Cooperation Foundation, Research Period: March 01, 2024 to February 28, 2025) and Bio-Medical Technology Development (R&D) of the Ministry of Science and ICT (Project Identification No.: RS-2025-02309992, Project Management Institution: National Research Foundation of Korea, Research Project Title: Development of Brain Neural Network Control and Monitoring Technology Based on Multifunctional Neural Interface Technology, Project Execution Institution: Kyung Hee University University-Industry Cooperation Foundation, Research Period: April 01, 2025 to December 31, 2027).

Meanwhile, the government of the Republic of Korea providing the project has no property interest in all aspects of the present disclosure.

### BACKGROUND

A medical implant device refers to a medical device inserted into a human body to treat a specific disease or to replace a body organ lost due to a disease or an accident. The medical implant device performs various medical functions such as cardiac pacing, nerve stimulation, and drug delivery, and when power supply to the device is stopped, a fatal problem such as cardiac arrest may occur.

Accordingly, technology for stably supplying power to the medical implant devices is very important, and wireless power transfer (WPT) technology has emerged as a solution for this problem.

Conventional WPT technology includes technology using radio frequency (RF) and technology using ultrasound, however, in case of the WTP using the RF, that is, an inductive coupling method in which two coils are aligned to transfer energy, while it ensures high power transfer efficiency, it generates a large amount of heat, and thus there is a problem in transferring energy into the human body.

FIG. 1A is a view showing the conventional WPT technology using ultrasound. In addition, FIG. 1B is a view showing conventional ultrasound and biointerface-based energy harvesting technology. Referring to FIG. 1A and FIG. 1B, in case of the WPT using ultrasound, there is no heat generation problem, but it is sensitive to an alignment error, so that when the medical implant device and an external power transmitter are not aligned, not only is the power transfer efficiency significantly reduced, but it is also vulnerable to a medium change, and thus there is a problem of exhibiting different power transfer efficiencies depending on a medium that exists therebetween.

Accordingly, a wireless power transfer apparatus that is not only robust to heat generation but also has low sensitivity to the alignment error and can maintain consistent power transfer efficiency under various medium conditions is required.

### SUMMARY

### TECHNICAL PROBLEM

The present disclosure is directed to providing a triboelectric nanogenerator that may suppress heat generation and stably supply power regardless of a medium by using a polymer material that responds to a magnetic field, and a wireless power transfer apparatus using the same.

In addition, the present disclosure is directed to providing a triboelectric nanogenerator that may maintain high power supply efficiency even in a misaligned state by reducing misalignment sensitivity by including a polymer material layer with a three-dimensional (3D) structure, and a wireless power transfer apparatus using the same.

In addition, the present disclosure is directed to providing a triboelectric nanogenerator having a high current value by using a plurality of electrodes and a plurality of polymer materials, and a wireless power transfer apparatus using the same.

The objects of the present specification are not limited to the above-mentioned objects, and other objects and advantages of the present specification that are not mentioned can be understood by the following description and will be more clearly understood by the embodiments of the present specification. In addition, it will be readily apparent that the objects and advantages of the present specification can be achieved by the means and combinations thereof set forth in the claims.

A triboelectric nanogenerator according to an embodiment of the present disclosure may include an electrode, a first polymer material layer with a three-dimensional (3D) structure, a second polymer material layer attached to at least a part of the first polymer material layer and in contact or not in contact with the electrode depending on an external magnetic field by including magnetic particles, and a spacer provided between the electrode and the first polymer material layer to form a predetermined separation distance, wherein a voltage peak may be generated when the second polymer material layer is in contact with the electrode.

In addition, in an embodiment of the present disclosure, the electrode may include a top electrode and a bottom electrode provided at a position corresponding to the top electrode based on the first polymer material layer, the spacer may include an upper spacer provided between the top electrode and the first polymer material layer and a lower spacer provided between the bottom electrode and the first polymer material layer, and the second polymer material layer may include an upper polymer material that is disposed to be in contact with an upper surface of the first polymer material layer and a lower polymer material that is disposed to be in contact with a lower surface of the first polymer material layer.

In addition, in an embodiment of the present disclosure, the upper polymer material may be in contact with the top electrode in response to the external magnetic field, and the lower polymer material may be in contact with the bottom electrode in response to the external magnetic field.

In addition, in an embodiment of the present disclosure, the second polymer material layer may be composed of a polydimethylsiloxane (PDMS) composite that is a polymer compound.

In addition, in an embodiment of the present disclosure, the second polymer material layer may be composed of a PDMS composite that includes neodymium magnetic particles (NdFeB) and barium titanate particles (BaTiO₃).

In addition, in an embodiment of the present disclosure, the first polymer material layer may be composed of polyimide (PI) that is a polymer material.

In addition, in an embodiment of the present disclosure, the first polymer material layer may have a 3D serpentine structure with a 3D uneven shape.

In addition, in an embodiment of the present disclosure, the first polymer material layer may include a first part in a central region, a second part that surrounds the first part and forms an outer periphery, and a third part that is formed in an uneven shape to radially connect the first part and the second part.

In addition, in an embodiment of the present disclosure, at least a part of the second part may be cut at a predetermined interval to be formed in a form in which a length of the third part is stretched.

In addition, in an embodiment of the present disclosure, a wireless power transfer apparatus may include an electromagnet that applies a magnetic field and a triboelectric nanogenerator that is driven in response to the magnetic field and wirelessly transfers generated power to a medical implant device, wherein the triboelectric nanogenerator may include a second polymer material layer attached to at least a part of a first polymer material layer and in contact or not in contact with an electrode depending on an external magnetic field by including magnetic particles and a spacer that is provided between the electrode and the first polymer material layer to form a predetermined separation distance, wherein a voltage peak is generated when the second polymer material layer is in contact with the electrode.

A triboelectric nanogenerator and a wireless power transfer apparatus using the same according to an embodiment of the present disclosure can suppress heat generation and stably supply power regardless of a medium by using a polymer material that responds to a magnetic field.

In addition, the triboelectric nanogenerator and the wireless power transfer apparatus using the same can maintain high power supply efficiency even in a misaligned state by reducing misalignment sensitivity by including a polymer material layer with a three-dimensional (3D) structure.

In addition, the triboelectric nanogenerator and the wireless power transfer apparatus using the same can have a high current value by using a plurality of electrodes and a plurality of polymer materials.

### BRIEF DESCRIPTION OF THE DRAWINGS

FIG. 1A is a view showing conventional wireless power transfer technology using ultrasound.

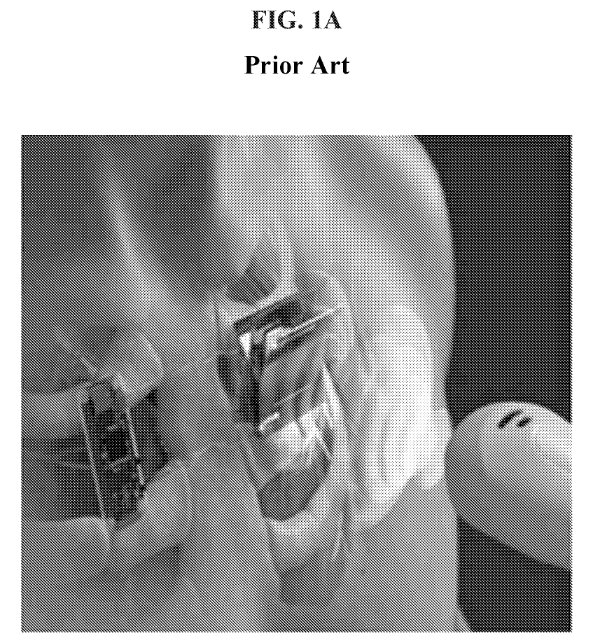

FIG. 1B is a view showing conventional ultrasound and biointerface-based energy harvesting technology.

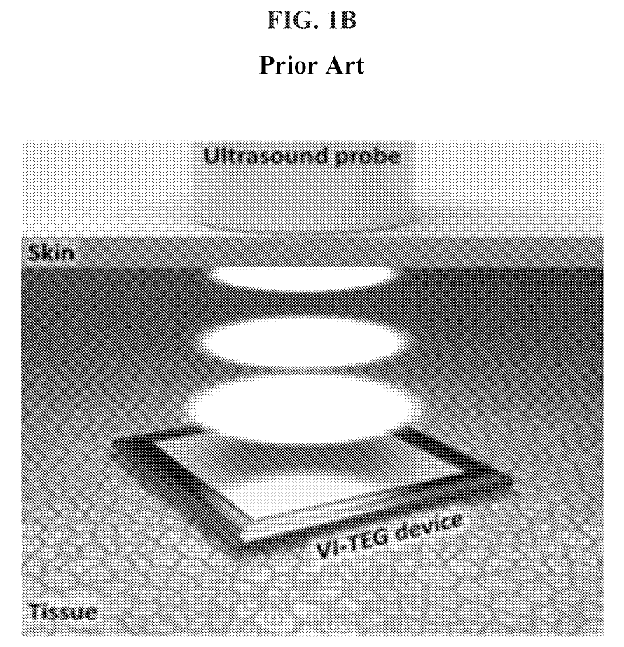

FIG. 2 is a usage state view of a wireless power transfer apparatus including a triboelectric nanogenerator according to various embodiments of the present disclosure.

FIG. 3 is an exploded perspective view of the triboelectric nanogenerator according to various embodiments of the present disclosure.

FIG. 4A is a view showing a state of a first polymer material layer in an initial state in which no external magnetic field is applied in an embodiment of the present disclosure.

FIG. 4B is a view showing a state in which an attractive force is applied to the first polymer material layer in an embodiment of the present disclosure.

FIG. 4C is a view showing a state in which a repulsive force is applied to the first polymer material layer in an embodiment of the present disclosure.

FIG. 5A is a graph showing a voltage peak according to a state of the first polymer material layer in an embodiment of the present disclosure.

FIG. 5B is an enlarged view of the voltage peak according to the state of the first polymer material layer in an embodiment of the present disclosure.

FIG. 6A is a view showing a single electrode rectifier circuit and voltage waveform conversion in an embodiment of the present disclosure.

FIG. 6B is a view showing a multi-electrode rectifier circuit and voltage waveform conversion in an embodiment of the present disclosure.

FIG. 6C is a view showing a time versus current graph of a triboelectric nanogenerator including one electrode under a 1 kΩ condition in an embodiment of the present disclosure.

FIG. 6D is an enlarged view of the time versus current graph of the triboelectric nanogenerator including one electrode under the 1 kΩ condition in an embodiment of the present disclosure.

FIG. 6E is a view showing a time versus current graph of a triboelectric nanogenerator including two electrodes under the 1 kΩ condition in an embodiment of the present disclosure.

FIG. 6F is an enlarged view of the time versus current graph of the triboelectric nanogenerator including two electrodes under the 1 kΩ condition in an embodiment of the present disclosure.

FIG. 7A is a view showing a three-dimensional (3D) structure in which a second part of the first polymer material layer is not broken in an embodiment of the present disclosure.

FIG. 7B is a view showing a 3D structure in which the second part of the first polymer material layer is broken and stretched in an embodiment of the present disclosure.

FIG. 8A is a view showing maximum strain concentration according to strain distribution generated when the first polymer material layer is actuated downward in an embodiment of the present disclosure.

FIG. 8B is an enlarged view of a maximum strain concentration region when the first polymer material layer is actuated downward in an embodiment of the present disclosure.

FIG. 8C is a view showing maximum strain concentration according to strain distribution generated when the first polymer material layer is actuated upward in an embodiment of the present disclosure.

FIG. 8D is a view showing an enlarged view of a maximum strain concentration region when the first polymer material layer is actuated upward in an embodiment of the present disclosure.

FIG. 9 is a graph showing power transfer efficiency according to lateral misalignment between a triboelectric nanogenerator and an electromagnet in an embodiment of the present disclosure.

FIG. 10 is a graph showing power transfer efficiency according to angular misalignment between the triboelectric nanogenerator and the electromagnet in an embodiment of the present disclosure.

FIG. 11 is a graph showing characteristics of a second polymer material layer of the present disclosure.

FIG. 12A is a result of testing an influence depending on a medium of the wireless power transfer apparatus according to an embodiment of the present disclosure.

FIG. 12B is a view showing an actual measurement environment in the medium of the wireless power transfer apparatus according to an embodiment of the present disclosure.

### DETAILED DESCRIPTION OF THE EMBODIMENTS

Hereinafter, exemplary embodiments according to the present disclosure will be described in detail with reference to the content described in the attached drawings. However, the present disclosure is not restricted or limited by the exemplary embodiments. Unless otherwise defined, all terms (including technical and scientific terms) used herein are to be used with a meaning commonly understood by those having ordinary skill in the art to which this disclosure pertains, but this may vary depending on the intention of those skilled in the art, case law, or emergence of new technologies, etc.

In addition, terms defined in a commonly used dictionary are not to be interpreted ideally or excessively unless clearly and specifically defined otherwise. In a specific case, there are terms that the applicant has arbitrarily selected, and in this case, their meanings will be described in detail in the corresponding description part. Accordingly, the terms used in herein should be defined based on the meaning of the terms and the overall content of the present disclosure, rather than simply the names of the terms.

When it is said throughout this specification that a part “includes” a certain component, this does not exclude other components unless otherwise stated, but means other components may be further included. In addition, the singular forms used herein also include the plural forms unless specifically stated otherwise. In addition, the expression “at least one of a, b, and/or c” described throughout the present specification may encompass “a alone”, “b alone”, “c alone”, “a and b”, “a and c”, “b and c”, or “all of a, b, and c”.

Meanwhile, terms such as “first and/or second” used herein may be used to describe various components, but they are only used for the purpose of distinguishing one component from another component, and are not intended to be limited to the components referred to by the terms. For example, without departing from the scope of the present disclosure, the first component may be named as the second component, and the second component may also be named as the first component.

Hereinafter, various embodiments of the present disclosure will be described in detail with reference to the attached drawings. In describing the embodiments, a description of technical contents that are well known in the technical field to which the present disclosure pertains and are not directly related to the present disclosure will be omitted. This is to convey the gist of the present disclosure more clearly without obscuring the same by omitting unnecessary explanation. For the same reason, some components in the attached drawings are exaggerated, omitted, or schematically shown. In addition, size of each component does not entirely reflect its actual size. In the present specification, like reference numerals may refer to like or corresponding components throughout.

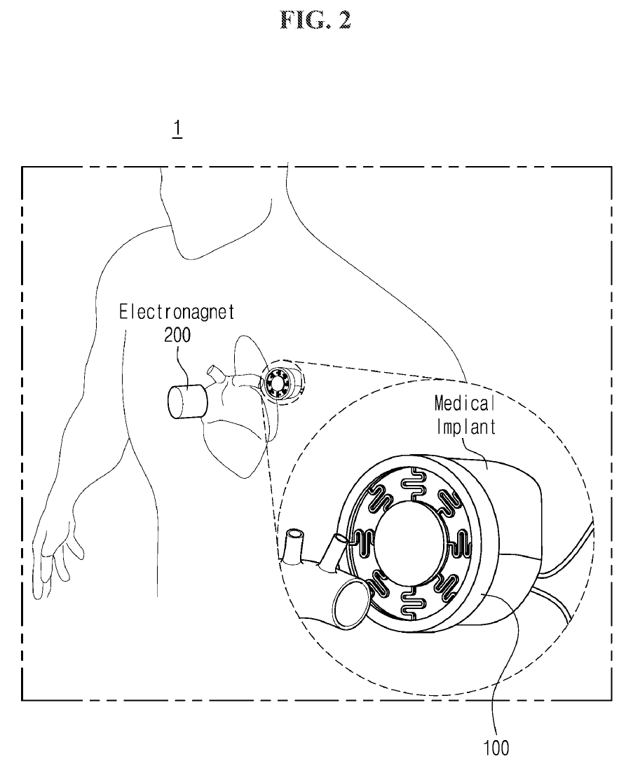

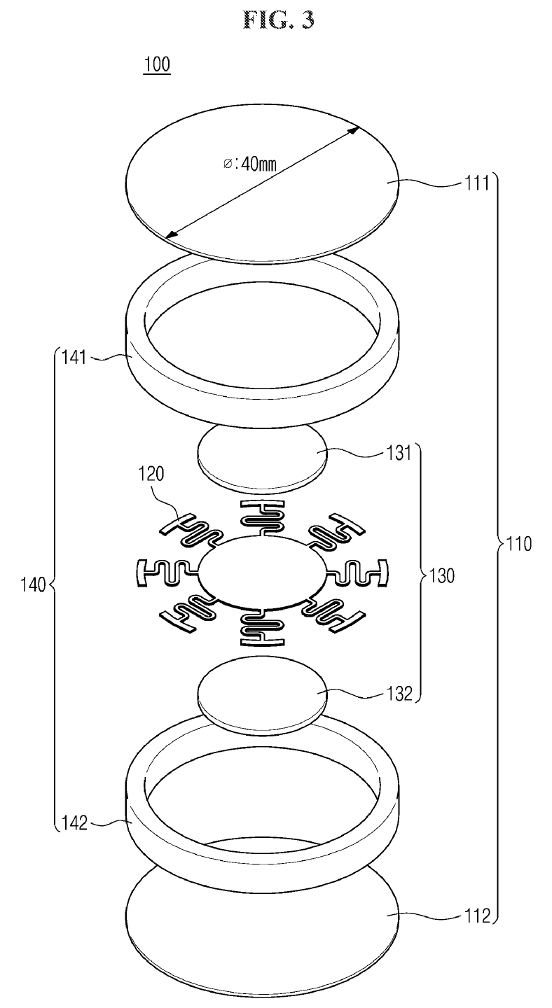

FIG. 2 is a usage state view of a wireless power transfer apparatus including a triboelectric nanogenerator according to various embodiments of the present disclosure, and FIG. 3 is an exploded perspective view of the triboelectric nanogenerator according to various embodiments of the present disclosure.

A triboelectric nanogenerator 100 is a device that is inserted into a human body as shown in FIG. 2 and generates power in response to a magnetic field applied from an external electromagnet 200. That is, as the triboelectric nanogenerator 100 of the present disclosure is driven on the basis of the external magnetic field, it is also referred to as a magnetically-actuated triboelectric nanogenerator (MA-TENG). The triboelectric nanogenerator 100 may be formed integrally with a medical implant device or formed separately from the medical implant device, and power generated from the triboelectric nanogenerator 100 is supplied to the medical implant device to maintain power so that the medical implant device is not discharged. Meanwhile, as the triboelectric nanogenerator 100 of the present disclosure is driven on the basis of the external magnetic field, stable power generation and supply are possible regardless of a medium through characteristics of the magnetic field that it is not affected by the medium.

Referring to FIG. 3, the triboelectric nanogenerator 100 may include an electrode 110, a first polymer material layer 120, a second polymer material layer 130, and a spacer 140.

The electrode 110 is a terminal that allows current to flow to the inside of the triboelectric nanogenerator 100 or discharge the same, may be a copper electrode with a diameter of 40 mm and high thermal conductivity, and may include a top electrode 111 and a bottom electrode 112. The top electrode 111 is disposed at an upper end based on the first polymer material layer 120, and the bottom electrode 112 is disposed at a lower end based on the first polymer material layer 120 to form a symmetrical structure with the top electrode 111.

The first polymer material layer 120 forms a three-dimensional (3D) structure composed of polyimide (PI) that is a polymer material. In detail, the first polymer material layer 120 may be a 3D serpentine structure with a 3D uneven shape and at least a part of the first polymer material layer 120 may flow upward or downward through this. The detailed structure and operation of the 3D serpentine will be described later.

The second polymer material layer 130 is attached to at least a part of the first polymer material layer 120 and may be composed of, for example, a polydimethylsiloxane (PDMS) composite that is a polymer material. In addition, in various embodiments, the second polymer material layer 130 may additionally include neodymium magnetic particles (NdFeB) and barium titanate particles (BaTiO₃) in the PDMS composite. In the embodiment, as the second polymer material layer 130 includes magnetic particles, the second polymer material layer 130 may respond to the magnetic field applied from outside and may be in contact with the electrode 110 by application of an attractive force or a repulsive force. In this case, static electricity is charged on a surface of the electrode 110 by contact, and thus energy may be harvested through a generated potential difference.

In addition, the second polymer material layer 130 may be provided in plural and include an upper polymer material 131 attached to an upper surface of the first polymer material layer 120 and a lower polymer material 132 attached to a lower surface of the first polymer material layer 120.

As the second polymer material layer 130 is attached to the upper and lower surfaces of the first polymer material layer 120, respectively, it may flow in both upper and lower directions rather than in one direction and may be in contact with any one of the top electrode 111 or the bottom electrode 112. In this case, it is certain that whether the second polymer material layer 130 will be in contact with the top electrode 111 or the bottom electrode 112 is determined by a polarity of the magnetic field applied from the outside.

The spacer 140 is provided between the electrode 110 and the first polymer material layer 120 to maintain a predetermined separation distance between the electrode 110 and the first polymer material layer 120. The spacer 140 may include an upper spacer 141 provided between the top electrode 111 and the first polymer material layer 120 and a lower spacer 142 provided between the bottom electrode 112 and the first polymer material layer 120, and may secure an internal space for the second polymer material layer 130 to flow upward or downward through the upper spacer 141 and the lower spacer 142.

Meanwhile, the spacer 140 may be formed of a 3mm thick acrylic material, but is not necessarily limited thereto, and may include all materials that do not cause current flow due to very low electric conductivity.

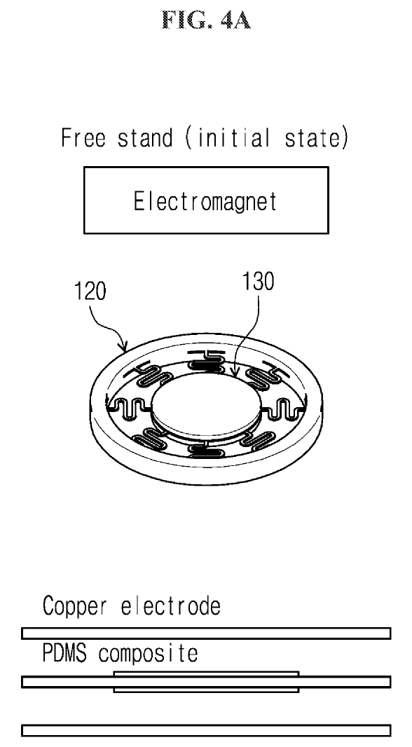

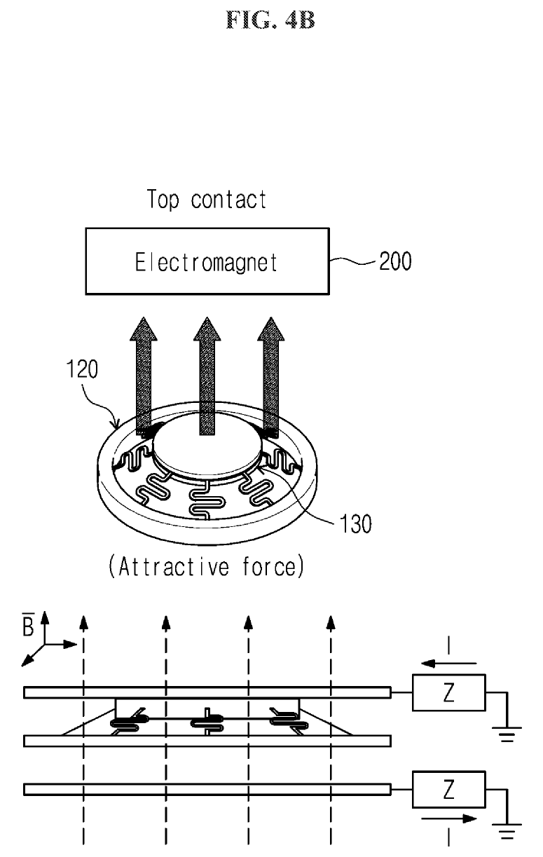

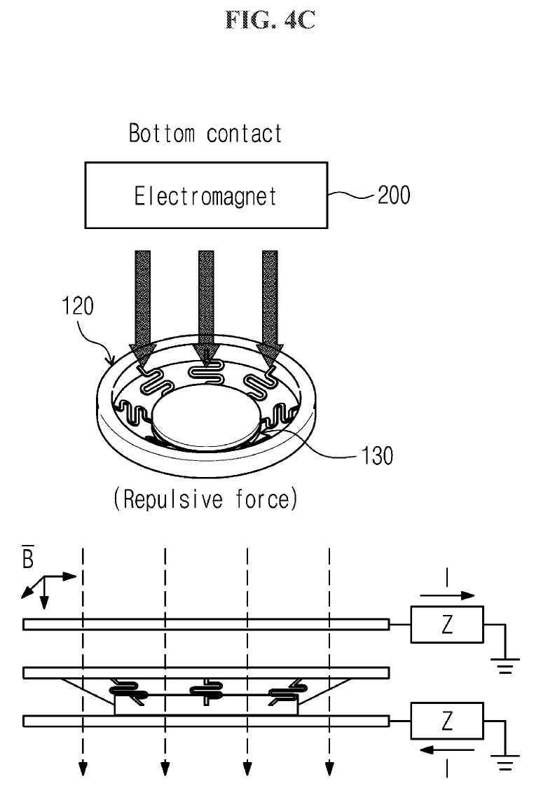

FIG. 4A is a view showing a state of a first polymer material layer in an initial state in which no external magnetic field is applied in an embodiment of the present disclosure, FIG. 4B is a view showing a state in which an attractive force is applied to the first polymer material layer in an embodiment of the present disclosure, and FIG. 4C is a view showing a state in which a repulsive force is applied to the first polymer material layer in an embodiment of the present disclosure.

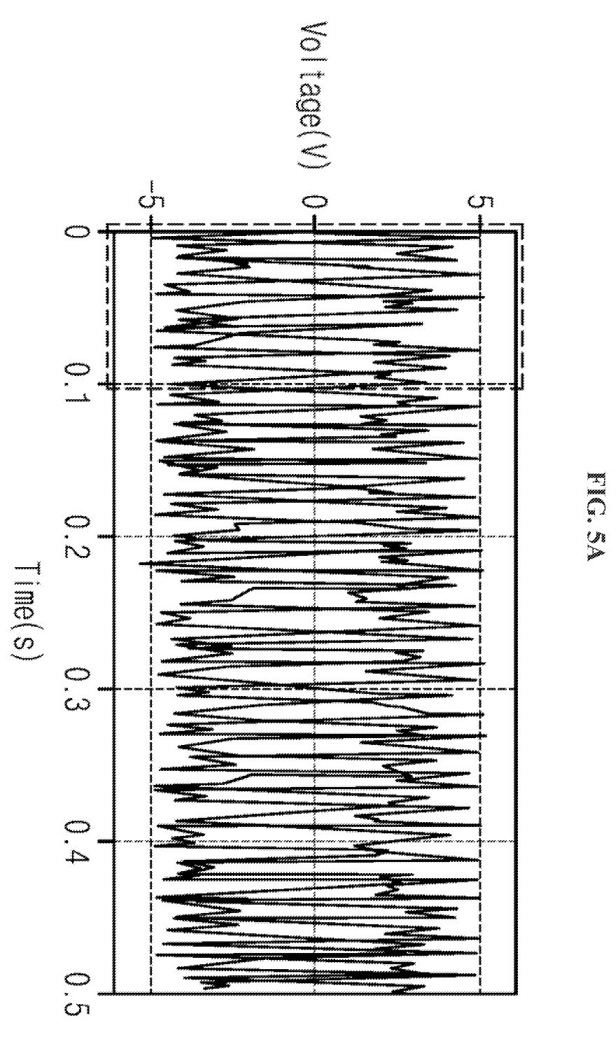

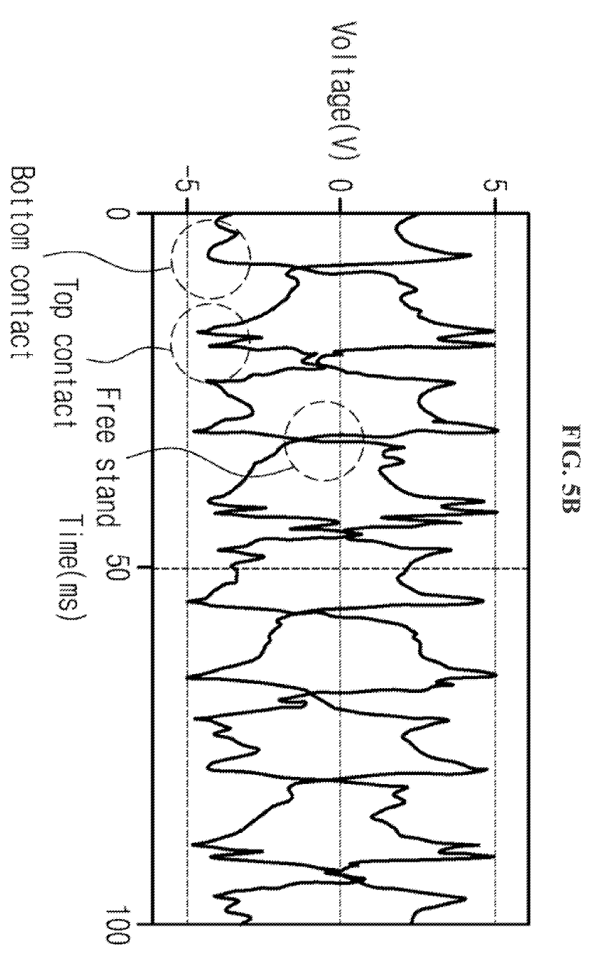

FIG. 5A is a graph showing a voltage peak according to a state of the first polymer material layer in an embodiment of the present disclosure, and FIG. 5B is an enlarged view of the voltage peak according to the state of the first polymer material layer in an embodiment of the present disclosure.

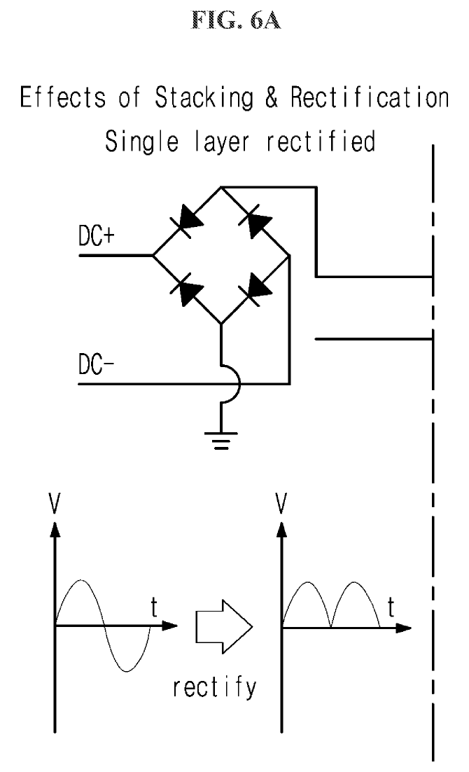

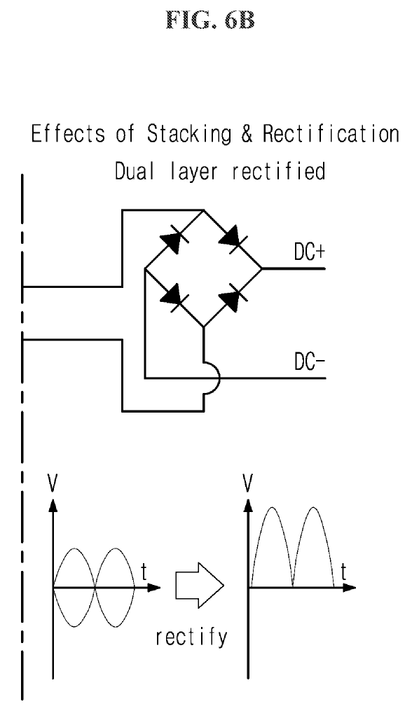

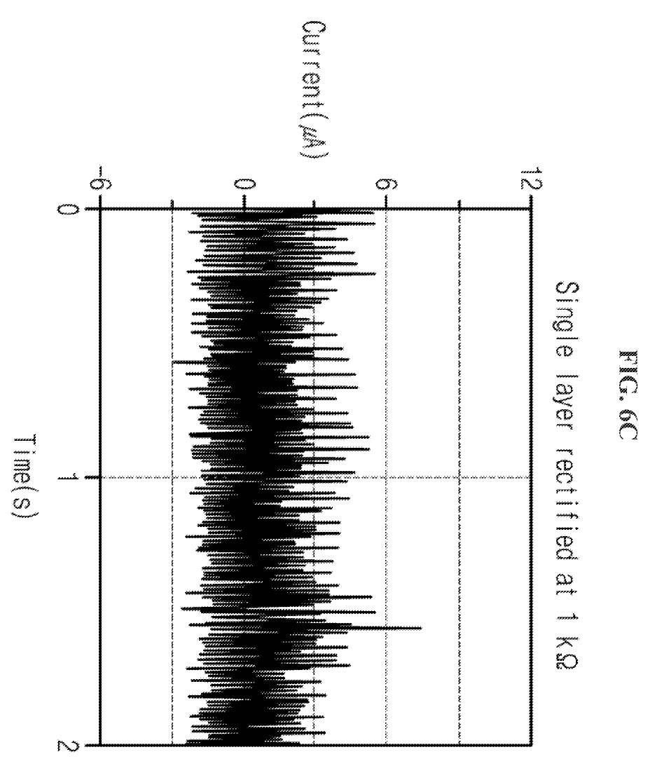

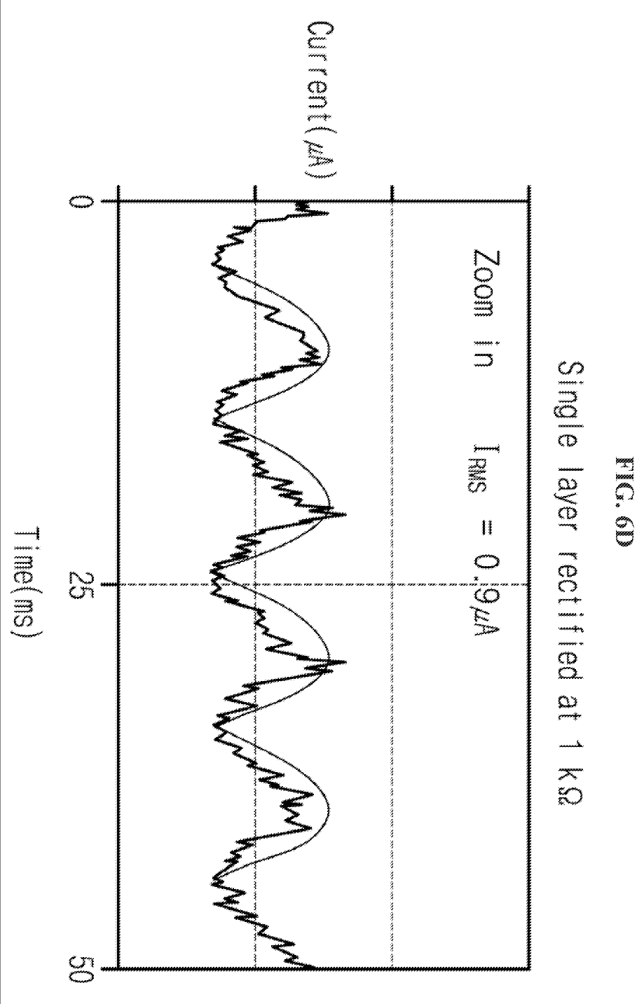

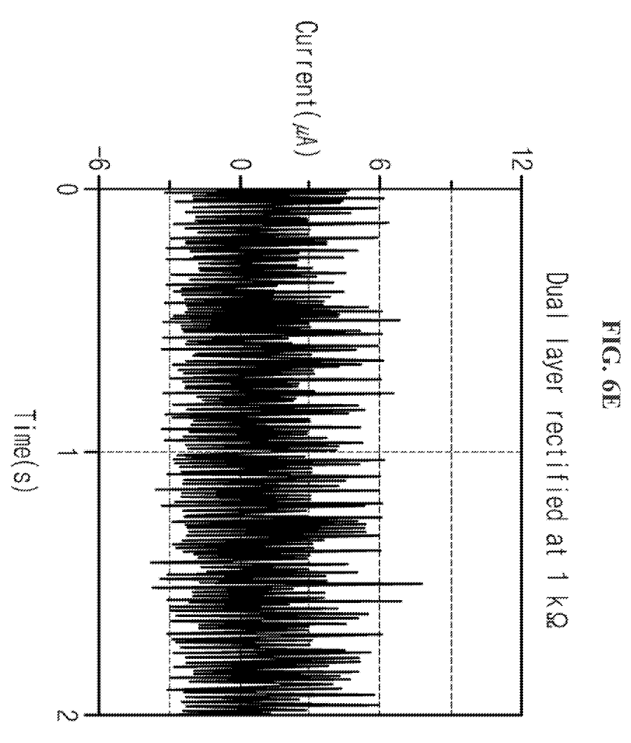

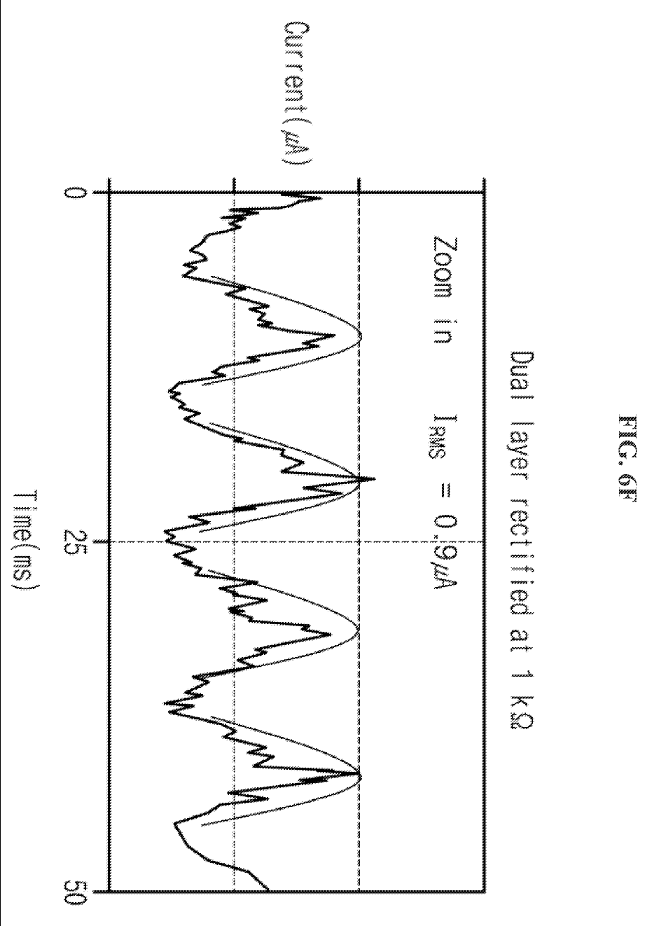

FIG. 6A is a view showing a single electrode rectifier circuit and voltage waveform conversion in an embodiment of the present disclosure, and FIG. 6B is a view showing a multi-electrode rectifier circuit and voltage waveform conversion in an embodiment of the present disclosure. FIG. 6C is a view showing a time versus current graph of one electrode rectifier circuit under a 1 kΩ condition in an embodiment of the present disclosure, FIG. 6D is an enlarged view of the time versus current graph of the one electrode rectifier circuit under the 1 kΩ condition in an embodiment of the present disclosure, FIG. 6E is a view showing a time versus current graph of two electrode rectifier circuit under the 1 kΩ condition in an embodiment of the present disclosure, and FIG. 6F is an enlarged view of the time versus current graph of the two electrode rectifier circuit under the 1 kΩ condition in an embodiment of the present disclosure. Hereinafter, the triboelectric nanogenerator will be described with reference to FIG. 4A to FIG. 6F.

Referring to FIG. 4A, in an initial state in which no external magnetic field is applied, the first polymer material layer 120 maintains neutrality between the top electrode 111 and the bottom electrode 112, forms an interval of a predetermined distance through the spacer 140, and is not in contact with the top electrode 111 and the bottom electrode 112.

In this case, when an external magnetic field generated by the electromagnet 200 is applied to the first polymer material layer 120, a state of contact is maintained with either the top electrode 111 or the bottom electrode 112 by the second polymer material layer 130 attached to the first polymer material layer 120. Specifically, as shown in FIG. 4B, when the attractive force is applied, the first polymer material layer 120 is in contact with the top electrode 111 (top contact), and as shown in FIG. 4C, when the repulsive force is applied, the first polymer material layer 120 is in contact with the bottom electrode 112 (bottom contact). As such, the attractive force and the repulsive force generated from the electromagnet may be determined according to a direction of current flowing in the electromagnet.

In summary, the first polymer material layer 120 alone does not respond to the magnetic field, but responds to the magnetic field as the second polymer material layer 130 is attached, and is in contact with the electrode 110 through the attractive force or the repulsive force. In addition, a potential difference is generated by contact with the electrode 110, thereby enabling power to be transferred to the medical implant device.

In addition, referring to FIG. 5A, when no external force is applied as in FIG. 4A, no voltage peak value is exhibited, but when the first polymer material layer 120 is in contact with the top electrode 111 as in FIG. 4B or when the first polymer material layer 120 is in contact with the bottom electrode 112 as in FIG. 4C, different voltage peak values are generated, and it may be confirmed through FIG. 5B that the voltage peaks appear alternately on the graph as the direction of current flow inside the electromagnet changes periodically and the polarity changes.

Accordingly, as shown in FIG. 6A to FIG. 6F, since two electrodes are included in one triboelectric nanogenerator 100, a current value of more than twice may be output. That is, when one electrode is included, an average current value of 0.9 μA was exhibited, but when configured as one rectifier circuit including two electrodes, an average current value of 1.9 μA was exhibited. Through this, a low current to a high voltage, which was a disadvantage of the triboelectric nanogenerator 100, may be solved.

Specifically, FIG. 6A is a view showing a single electrode rectifier circuit and voltage waveform conversion, and FIG. 6B is a view showing a multi-electrode rectifier circuit and voltage waveform conversion.

FIG. 6C is a view showing a time versus current graph of a triboelectric nanogenerator 100 including one electrode under a 1 kΩ condition. FIG. 6D is an enlarged view of the time versus current graph of the triboelectric nanogenerator 100 including one electrode under the 1 kΩ condition. Referring to FIG. 6D, it may be confirmed that the triboelectric nanogenerator 100 including one electrode exhibits an average current value of 0.9 μA.

FIG. 6E is a view showing a time versus current graph of a triboelectric nanogenerator 100 including two electrodes under the 1 kΩ condition. FIG. 6F is an enlarged view of the time versus current graph of the triboelectric nanogenerator 100 including two electrodes under the 1 kΩ condition. Referring to FIG. 6F, it may be confirmed that the triboelectric nanogenerator 100 including two electrodes exhibits an average current value of 1.9 μA.

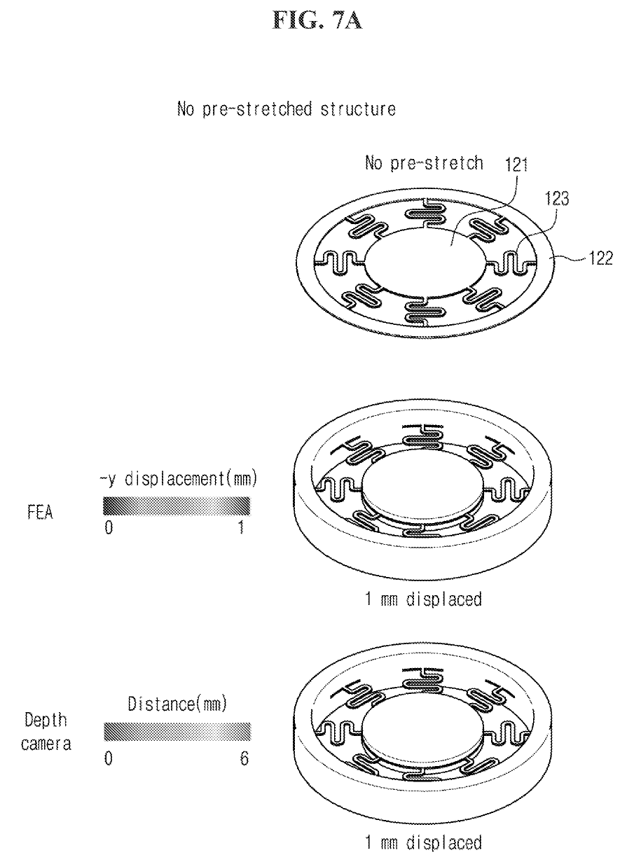

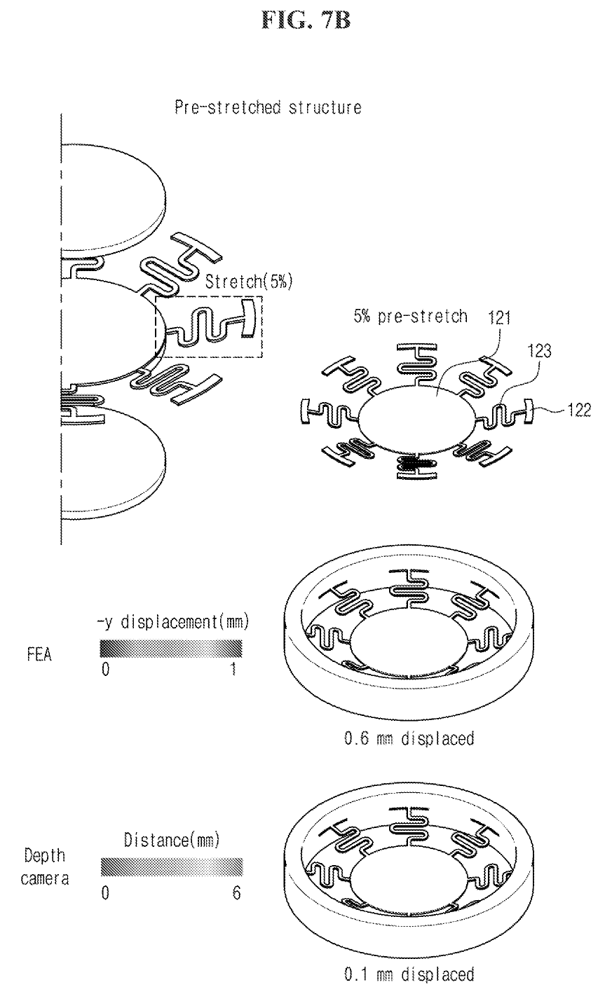

FIG. 7A is a view showing a 3D structure in which a second part of the first polymer material layer is not broken in an embodiment of the present disclosure, and FIG. 7B is a view showing a 3D structure in which the second part of the first polymer material layer is broken and stretched in an embodiment of the present disclosure.

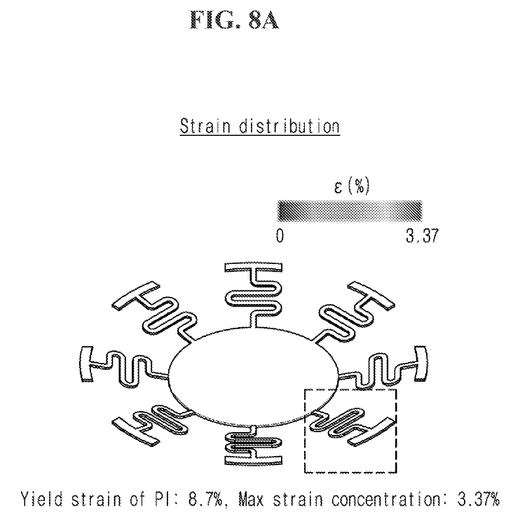

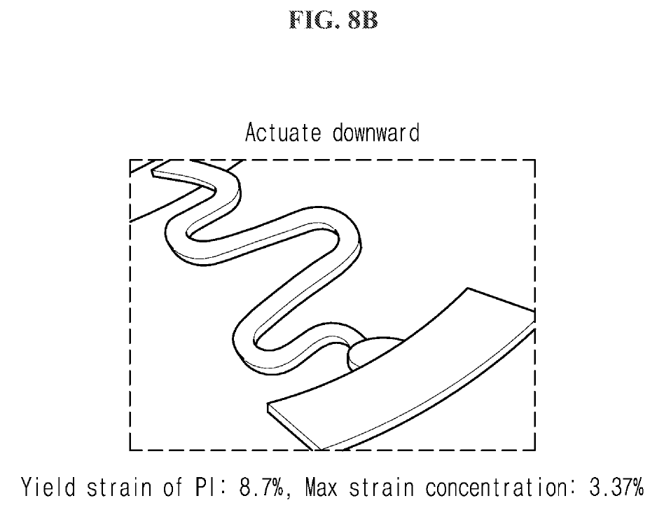

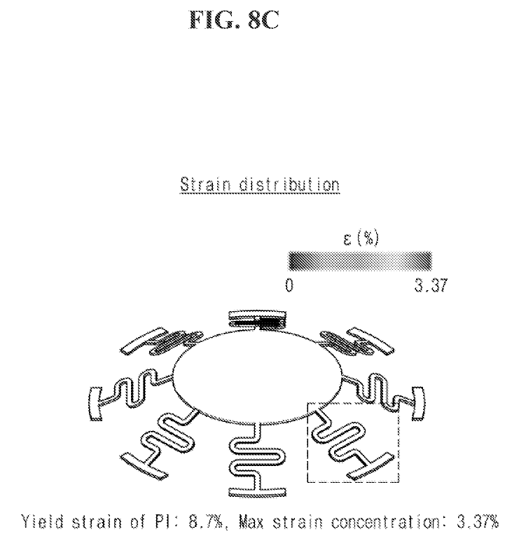

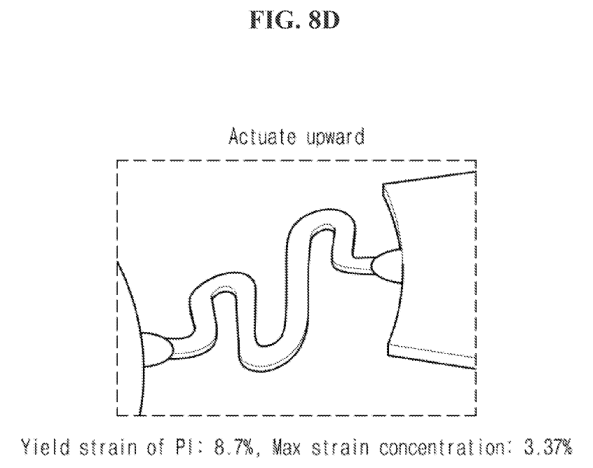

In addition, FIG. 8A is a view showing maximum strain concentration according to strain distribution generated when the first polymer material layer is actuated downward in an embodiment of the present disclosure, and FIG. 8B is an enlarged view of a maximum strain concentration region when the first polymer material layer is actuated downward in an embodiment of the present disclosure. In addition, FIG. 8C is a view showing maximum strain concentration according to strain distribution generated when the first polymer material layer is actuated upward in an embodiment of the present disclosure, and FIG. 8D is a view showing an enlarged view of a maximum strain concentration region when the first polymer material layer is actuated upward in an embodiment of the present disclosure.

As mentioned above, the first polymer material layer 120 forms a 3D structure composed of polyimide (PI) that is a polymer material, and in detail, the first polymer material layer 120 may form a 3D serpentine structure with a 3D uneven shape.

Referring to FIG. 7A and FIG. 7B, the first polymer material layer 120 with the 3D structure includes a first part 121, a second part 122, and a third part 123.

The first part 121 is a circular central region to which the second polymer material layer 130 is attached, and the upper polymer material 131 may be disposed on an upper surface thereof and the lower polymer material 132 may be disposed on a lower surface thereof. When the external magnetic field is applied, the first part 121 may flow upward or downward through the second polymer material layer 130 to be in contact with the top electrode 111 or the bottom electrode 112.

The second part 122 is a region that forms an outer periphery of the first polymer material layer 120 and is fixedly supported by the spacer 140 without flowing.

The third part 123 physically connects the first part 121 and the second part 122, but may be formed in an uneven shape so that the first part 121 may sufficiently flow upward or downward to stretch while maintaining appropriate tension between the first part 121 and the second part 122. In addition, the third part 123 may be formed radially in plural between the first part 121 and the second part 122.

As such, the first polymer material layer 120 includes the first part 121 that is the central region, the second part 122 that is an outer periphery region, and the third part 123 that connects the first part and the second part, thereby forming the 3D serpentine structure.

Meanwhile, as the second polymer material layer 130 is attached to the first part 121 of the first polymer material layer 120, a sagging phenomenon due to a load may occur. In various embodiments, in order to alleviate sagging, at least a part of the second part 122 may be cut at a regular interval to be formed in a form in which a length of the third part 123 is stretched (pre-stretched) as shown in FIG. 7. As the length of the third part 123 is stretched and a radius of the first polymer material layer 120 increases, a tensile force acting between the first part 121 and the second part 122 increases, thereby alleviating the sagging phenomenon. Here, a rate at which the third part 123 is stretched may be, for example, 5%.

Referring to FIG. 7A, in a case of a form (no pre-stretch) in which the second part 122 of the first polymer material layer 120 is not broken, a 1 mm sagging occurred as a result of a simulation, and when measured through a depth camera, the 1 mm sagging also occurred. On the other hand, referring to FIG. 7B, in a case of a form (pre-stretched) in which at least a part of the second part 122 is cut at a regular interval so that the third part 123 is stretched, a 0.6 mm sagging occurred as a result of a simulation, and when measured through the depth camera, a 0.1 mm sagging occurred, and thus it can be seen that a degree of sagging was reduced.

In addition, referring to FIG. 8A, a yield strain of PI is 8.7%, while maximum strain concentration that occurs when the first polymer material layer 120 is maximally deformed through downward actuation is 3.37% that is lower than the yield strain of PI, thereby indicating that the first polymer material layer 120 in the 3D serpentine structure of the present disclosure is a structure that is mechanically very stable while being easily deformable by the magnetic field. Here, an appearance in which the third part 123 is stretched downward may be specifically confirmed with reference to FIG. 8B.

In addition, referring to FIG. 8C, the yield strain of PI is 8.7%, while maximum strain concentration that occurs when the first polymer material layer 120 is maximally deformed through upward actuation is 3.37% that is lower than the yield strain of PI, thereby confirming again that the first polymer material layer 120 in the 3D serpentine structure of the present disclosure is a structure that is mechanically very stable while being easily deformable by the magnetic field. Here, an appearance in which the third part 123 is stretched upward may be specifically confirmed with reference to FIG. 8D.

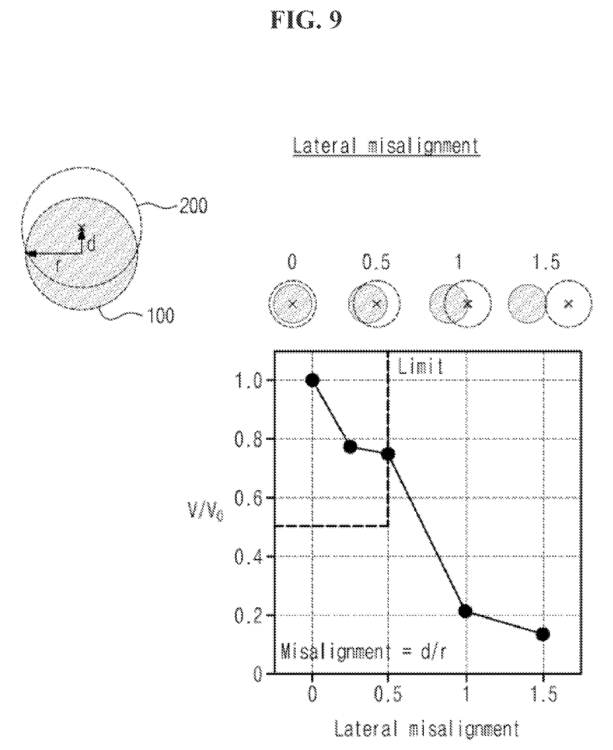

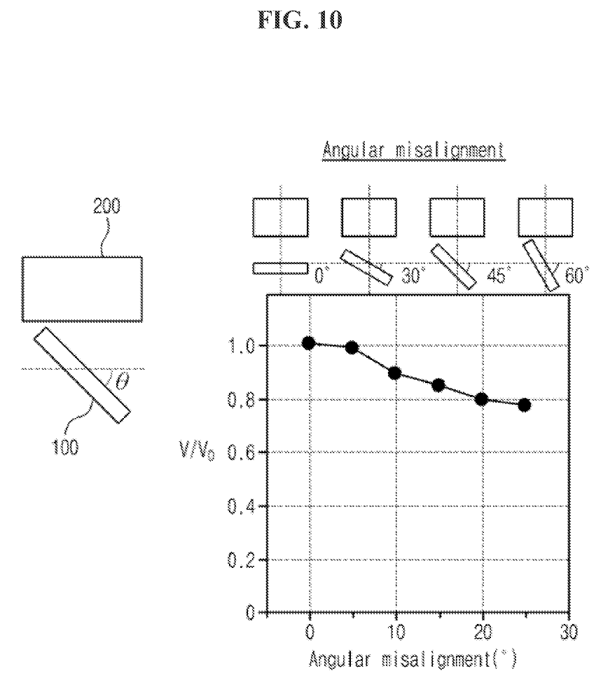

FIG. 9 is a graph showing power transfer efficiency according to lateral misalignment between a triboelectric nanogenerator and an electromagnet in an embodiment of the present disclosure, and FIG. 10 is a graph showing power transfer efficiency according to angular misalignment between the triboelectric nanogenerator and the electromagnet in an embodiment of the present disclosure.

In a case of the triboelectric nanogenerator 100, since it is inserted into a human body, it is very difficult to precisely align a center of the triboelectric nanogenerator 100 and a center of the electromagnet 200, and when the power transfer efficiency is reduced due to an alignment error, usability is greatly reduced. The triboelectric nanogenerator 100 of the present disclosure solves the problem of reduced power transfer efficiency due to misalignment in the triboelectric nanogenerator using conventional ultrasound by applying the above-mentioned 3D structure.

Referring to FIG. 9, when a distance between the center of the electromagnet 200 and the center of the triboelectric nanogenerator 100 is d and a radius of the triboelectric nanogenerator 100 is r, an x-axis of a graph represents a d/r value, and a y-axis represents the power transfer efficiency. When viewed in detail, it may be confirmed that even when an error of about 50% occurred and caused the misalignment, high efficiency of 0.8, that is, 80% or more is maintained and even when an error of 100% occurred and a degree of the misalignment is high, efficiency of 20% is maintained.

In addition, the triboelectric nanogenerator 100 may be moved according to movement inside the human body, thereby causing a change in position or a twist phenomenon. Referring to FIG. 10, the x-axis represents an angle θ formed by the triboelectric nanogenerator 100 and the electromagnet 200, and the y-axis represents the power transfer efficiency. When viewed in detail, it was shown that even an angular error of 25 degrees occurred and caused the misalignment, high efficiency of 0.8, that is, 80% or more was maintained.

As such, since the triboelectric nanogenerator 100 of the present disclosure has high power transfer efficiency even with the lateral misalignment and the angular misalignment, the medical implant device may be efficiently charged even when a user is moving or performing a daily activity, thereby greatly improving practicality of the medical device.

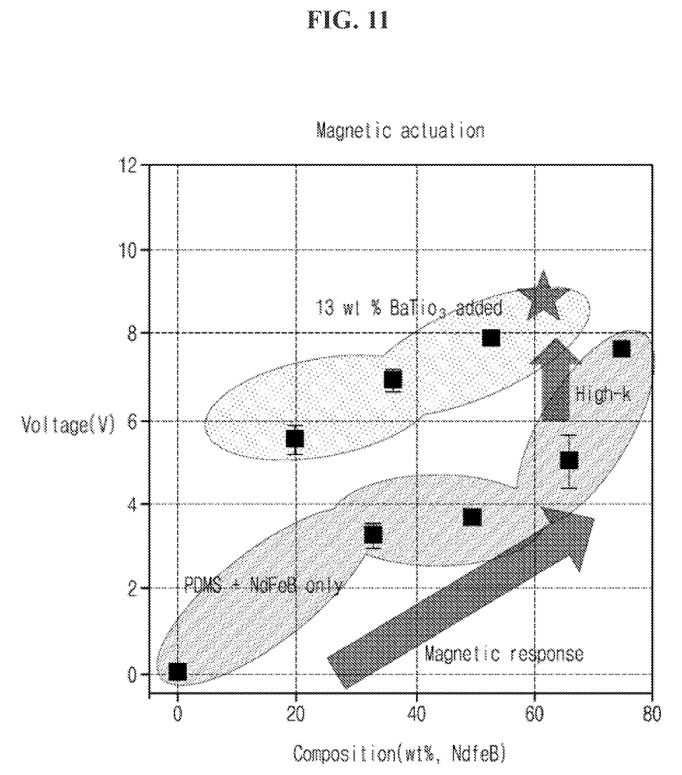

FIG. 11 is a graph showing characteristics of a second polymer material layer of the present disclosure.

The second polymer material layer 130 of the present disclosure may be the PDMS composite that is the polymer compound, and the neodymium magnetic particles (NdFeB) and the barium titanate particles (BaTiO₃) may be additionally added thereto.

First, when the neodymium magnetic particles (NdFeB) are added to the PDMS, it may respond to the magnetic field, thereby improving the magnetic response as a content (wt.%) of the neodymium magnetic particles (NdFeB) increases in the graph as shown in FIG. 11 and an output voltage continuously increases as the PDMS is in strong contact with the electrode. In the embodiment, it may be desirable for the neodymium magnetic particles (NdFeB) to be formed at the content of 75% or less.

Separately, when the barium titanate particles (BaTiO₃) are added to the PDMS, surface energy is reduced, thereby inducing an electrostatic charging phenomenon to occur smoothly. In addition, the dielectric constant is increased by adding the barium titanate particles (BaTiO₃) that are ferroelectric, and the output voltage may also increase proportionally.

In summary, the magnetic field reactivity is improved by adding the NdFeB particles, thereby improving the output voltage, and the dielectric constant is improved by adding the BaTiO₃ particles, thereby improving the output voltage.

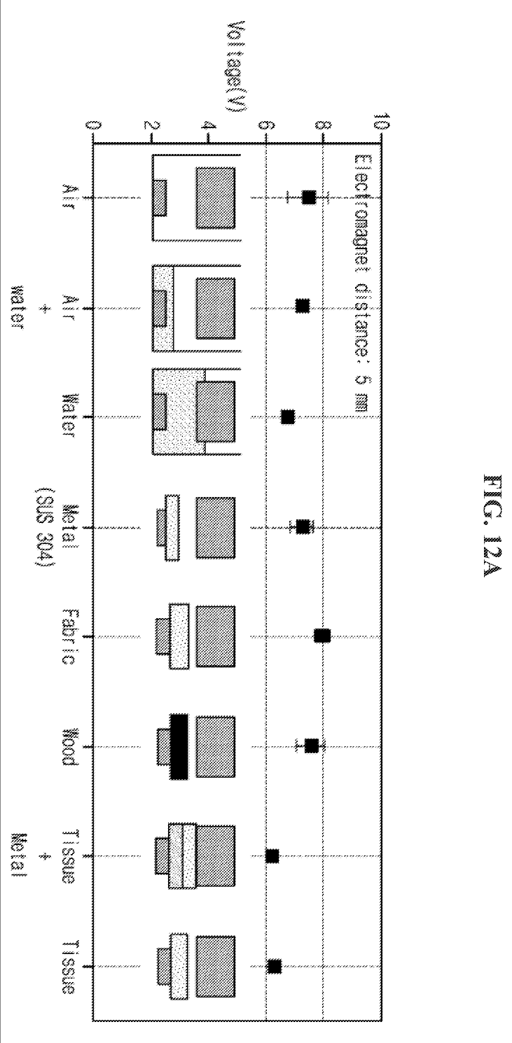

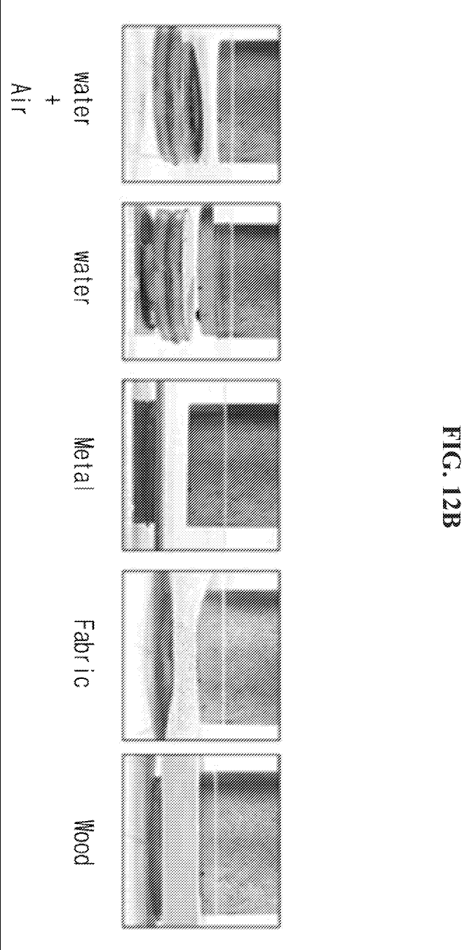

FIG. 12A is a result of testing an influence depending on a medium of the wireless power transfer apparatus according to an embodiment of the present disclosure. In addition, FIG. 12B is a view showing an actual measurement environment in the medium of the wireless power transfer apparatus according to an embodiment of the present disclosure.

In case of the ultrasound, the power transfer efficiency has very different values depending on which medium it passes through. In contrast, in case of the magnetic field, the dependence on the medium is remarkably low due to its characteristics.

Therefore, the power transfer efficiency depending on the medium was experimented using the wireless power transfer apparatus 1 of the present disclosure that transfer power on the basis of the magnetic field. The experiment was conducted on various media with different physical properties such as air, air/water, water, metal, fiber, wood, tissue/metal, and tissue, and as a result of the test, it may be seen that the triboelectric nanogenerator 100 of the present disclosure maintains similar power transfer efficiency regardless of the medium as shown in FIG. 12.

Although the present disclosure has been described with reference to the drawings as examples, it is obvious that the present disclosure is not limited to the embodiments and drawings disclosed herein, and that various modifications can be made by those having ordinary skill in the art within the scope of the technical idea of the present disclosure. In addition, even when the action effects of the configuration of the present disclosure were not explicitly described and explained while explaining the embodiments of the present disclosure, it is natural that the effects that can be predicted by the configuration should also be acknowledged.

What is claimed is:

1. A triboelectric nanogenerator comprising:
an electrode;
a first polymer material layer with a three-dimensional (3D) structure;
a second polymer material layer attached to at least a part of the first polymer material layer and in contact or not in contact with the electrode depending on an external magnetic field by including magnetic particles; and
a spacer provided between the electrode and the first polymer material layer to form a predetermined separation distance,
wherein a voltage peak is generated when the second polymer material layer is in contact with the electrode.

2. The triboelectric nanogenerator of claim 1, wherein the electrode includes a top electrode and a bottom electrode provided at a position corresponding to the top electrode based on the first polymer material layer,
the spacer includes an upper spacer provided between the top electrode and the first polymer material layer and a lower spacer provided between the bottom electrode and the first polymer material layer, and
the second polymer material layer includes an upper polymer material that is disposed to be in contact with an upper surface of the first polymer material layer and a lower polymer material that is disposed to be in contact with a lower surface of the first polymer material layer.

3. The triboelectric nanogenerator of claim 2, wherein the upper polymer material is in contact with the top electrode in response to the external magnetic field, and the lower polymer material is in contact with the bottom electrode in response to the external magnetic field.

4. The triboelectric nanogenerator of claim 1, wherein the second polymer material layer is composed of a polydimethylsiloxane (PDMS) composite that is a polymer compound.

5. The triboelectric nanogenerator of claim 4, wherein the second polymer material layer is composed of a PDMS composite that includes neodymium magnetic particles (NdFeB) and barium titanate particles (BaTiO₃).

6. The triboelectric nanogenerator of claim 1, wherein the first polymer material layer is composed of polyimide (PI) that is a polymer material.

7. The triboelectric nanogenerator of claim 1, wherein the first polymer material layer has a 3D serpentine structure with a 3D uneven shape.

8. The triboelectric nanogenerator of claim 7, wherein the first polymer material layer includes:
a first part in a central region;
a second part that surrounds the first part and forms an outer periphery; and
a third part that is formed in an uneven shape to radially connect the first part and the second part.

9. The triboelectric nanogenerator of claim 8, wherein at least a part of the second part is cut at a predetermined interval to be formed in a form in which a length of the third part is stretched.

10. A wireless power transfer apparatus comprising:
an electromagnet that applies a magnetic field; and
a triboelectric nanogenerator that is driven in response to the magnetic field and wirelessly transfers generated power to a medical implant device,
wherein the triboelectric nanogenerator includes:
a second polymer material layer attached to at least a part of a first polymer material layer and in contact or not in contact with an electrode depending on an external magnetic field by including magnetic particles; and
a spacer that is provided between the electrode and the first polymer material layer to form a predetermined separation distance,
wherein a voltage peak is generated when the second polymer material layer is in contact with the electrode.
